(19) United States
(12) Patent Application Publication
(10) Pub. No.: US 2003/0050519 A1
(43) Pub. Date: Mar. 13, 2003

(54) Method for decomposing plastic waste to produce fuel materials and equipment for the method

(76) Inventor: Ming-Chin Cheng, Changhua Hsien (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 09/949,576

(22) Filed: Sep. 11, 2001

(51) Int. Cl. C07C 4/00; C10B 21/20; C10B 57/00

(52) U.S. Cl. 585/241; 201/2.5; 202/99; 202/108

(57) ABSTRACT

A method for decomposing plastic waste to produce fuel materials makes the plastic waste decomposed by cracking and then inverted the plastic waste to be multiple fuel materials such as gas, gasoline, and diesel oil, and other byproducts such as hydrochloride acid and active carbon by fractionation. Whereby, the plastic waste is reused in an efficient and beneficial way.

### BACKGROUND OF THE INVENTION

1. Field of the Invention

The present invention relates to a method for decomposing plastic waste, and more particularly to a method that converts the plastic waste into multiple fuel materials by cracking thereby reducing an amount of plastic waste in a beneficial way. This invention also discloses equipment for this method.

2. Description of Related Art

Plastic is widely used of all kinds of products in our daily life and in considerable quantities because of its numerous advantages such as being cheap, light, easily shaped, and water-resisting. Therefore, plastic waste has become a major element in garbage. However, because most of the plastic waste does not decompose naturally in garbage dumps and creates toxic smoke after burning, the plastic waste is not environmentally friendly and causes very serious environmental problems. Recycling the plastic waste into other plastic products is a useful way to reduce the plastic waste, but this method is limited to thermoplastic materials and the recycled plastic product is of poor quality so that the recycling is not an efficient way to handle all plastic waste.

Therefore, the present invention has arisen to deal with all kinds of plastic waste in an efficient and beneficial way.

### SUMMARY OF THE INVENTION

The main objective of the present invention is to provide a method for decomposing plastic waste to produce multiple fuel materials and chemical reagents so as to reduce the amount of the plastic waste.

Further benefits and advantages of the present invention will become apparent after a careful reading of the detailed description with appropriate reference to the accompanying drawings.

### BRIEF DESCRIPTION OF THE DRAWINGS

FIG. 1 is a flow chart of a method for decomposing plastic waste to produce fuel materials; and

FIG. 2 is a schematic explanatory diagram showing a constitution of apparatus in according with the flow chart in FIG. 1.

### DETAILED DESCRIPTION OF THE INVENTION

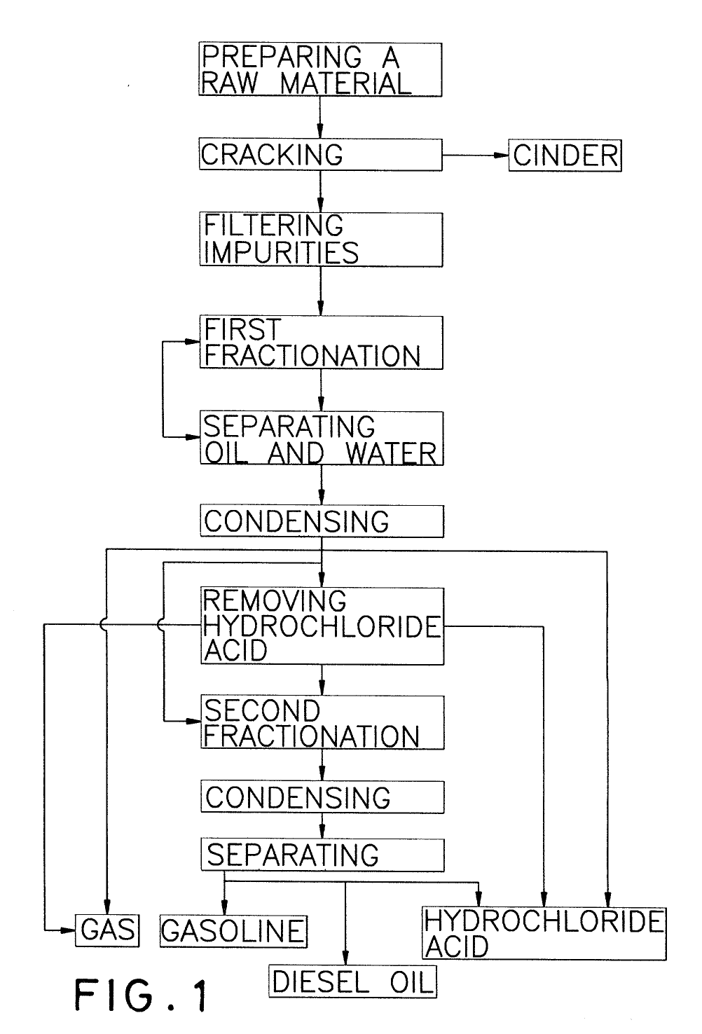

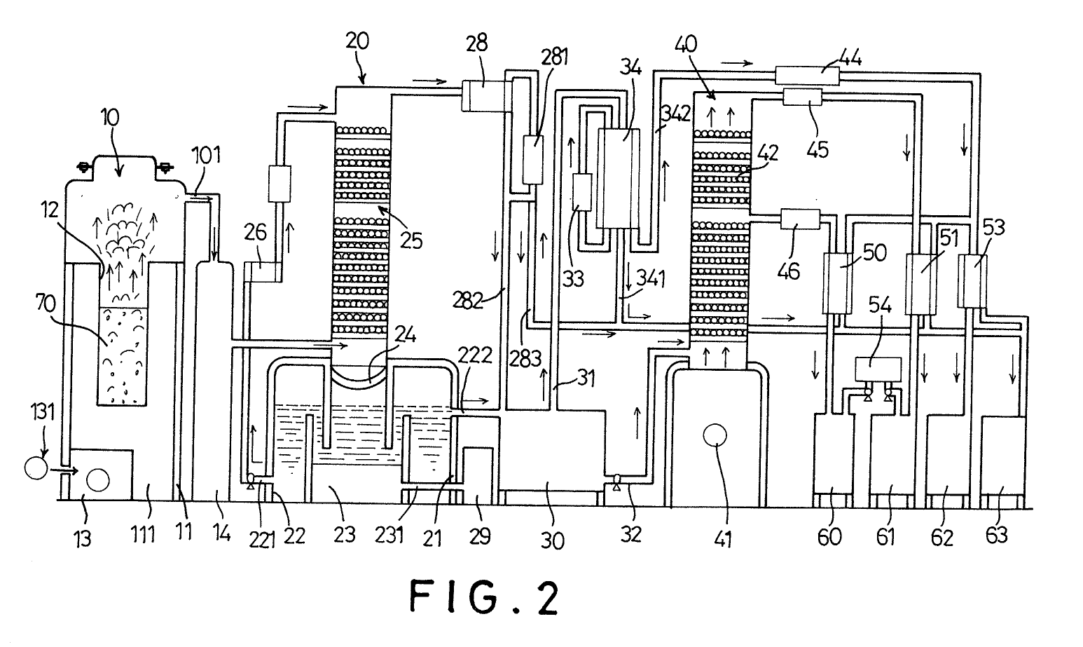

With reference to FIGS. 1 and 2, a method for decomposing plastic waste to produce fuel materials is composed of the following acts:

1. Preparing a raw material: the plastic waste made of polypropylene (PP), polystyrene (PS), polyurethane (PU), polyvinyl chloride (PVC), acrylonitrile-butadiene styrene (ABS resin), epoxy resin (EPS), and polyester (PE) . . . etc is mixed with catalyst and waste lube oils to compose the raw material.

2. Cracking: the raw material is inputted into a cracking device (10). The cracking device (10) comprises an outer tank (11), a heating tank (111) secured inside the outer tank (11), a heater (13) secured at a bottom of the outer tank (11) to heat up the cracking device (10), and a cracking tank (12) mounted on the outer tank (11) for receiving and cracking the raw material. A heat source (131) starts the heater (13) to produce heat to make the heating tank (111) heated up to 850° C. and the cracking tank (12) preferably heated up within a range 320-360° C. When the cracking device (10) operates, the raw material inside the cracking tank (12) is decomposed into cracked gas at high temperature and then the cracked gas escapes from a top tube (101) of the cracking device (10). Additionally, after cracking the raw material, cinder remains at a bottom of the cracking tank (12) wherein the cinder is purified and dedicated to compose of active carbon.

3. Filtering impurities: the cracked gas is guided into a filter tower (14) via the top tube (101). The filter tower (14) filters impurities such as particle and ash suspended in the cracked gas to avoid the impurities blocking the pipes of the following process and polluting the fuel materials.

4. First fractionation: after purifying by the filter tower (14), the cracked gas is sent into a first fractionating tower (20). The fractionating tower (20) comprises a water/oil separating tank (21), a first plurality of fractionating compartments (25) vertically mounted on the w/o separating tank (21), and a funnel (24) secured between the w/o separating tank (21) and the first plurality of fractionating compartments (25). The cracked gas is gradually cooled down from a top portion to a bottom portion with a temperature range from 360° C. to 80° C. Heavy gas oil with water are condensed and dropped along the funnel (24) to the w/o separating tank (21). Residuum gas of the cracked gas after fractionating flows into a condenser (28).

5. Separating oil and water: the heavy gas oil with water are stored in the w/o separating tank (21) and then separated into two different layers because of different specific gravities and undissolvable properties. The w/o separating tank (21) consists of an outer tank (22) and an inner tank (23) wherein the inner tank (23) has a wall lower than a wall of the outer tank (22). Therefore, an upper layer of heavy gas oil (containing a slight amount of water) in an upper layer will overflow from the inner tank (23) over the wall of the inner tank (23) to pour into the outer tank (22). A recycling pipe (221) communicates between a bottom portion of the outer tank (22) and a top portion of the first fractionating tower (20). The recycling pipe (221) combines with a pump (26) to pump up water in the bottom portion of the outer tank (22) to the first fractionating tower (20) so as to make the water re-fractionate. An oil outlet (222) communicates with the outer tank (22) at a high-liquid mark to drain heavy gas oil to a heavy gas oil tank (30). A water outlet (231) communicates the bottom portion of the inner tank (23) to drain water out to a water-storage tank (29).

6. Condensing: the residuum gas in the first fractionating tower (20) is released from the top of the first fractionating tower (20) and condensed in the condenser (28) to further separate heavy gas oil from the gas. Then, the heavy gas oil is drained into the heavy gas oil tank (30) and the gas flows into an extracting device (281) to extract hydrogen and chlorine from the gas. The hydrogen and the chlorine are composed and mixed with water to produce hydrochloride acid and then the hydrochloride acid is drained into a hydrochloride acid tank (63).

7. Removing hydrochloride acid: the heavy gas oil tank (30) stores the heavy gas oil and has a first pipe (31) with a pump to drain gas remaining in the heavy gas oil tank (30) to a first washing tower (34). The first washing tower (34) removes the hydrochloride acid remaining in gas by water and then restores the hydrochloride acid in the hydrochloride acid tank (63) via a forked tube (341) of the first washing tower (34). A recycling pump (33) compresses the gas to be recycled and re-purified. Additionally, gas after removing hydrochloride acid is guided into a gas tank (62) via a gas tube (342), wherein a vacuum pump (44) is secured on the gas tube (342) to push the gas forward to a second washing tower (53) so as to remove surplus hydrochloride acid from the gas.

8. Second fractionation: the heavy gas oil tank (30) has a second pipe (32) to transport the heavy gas oil to a second fractionating tower (40). The second fractionating tower (40) is composed of a heating furnace (41) to release heat and a second plurality of fractional compartments (42) mounted on the heating furnace (41). The heavy gas oil is partially vaporized and then fractionated in different fractional compartments (42) because multiple oils contained in the heavy gas oil each have different boiling points. Diesel gas is vaporized and separated at a high temperature from 100° C. to 220° C. and gasoline gas is vaporized and separated from 220° C. to330° C.

9. Condensing: the diesel gas and gasoline gas both in the vapor phase are condensed in a first cooler (45) and a second cooler (46) to liquidize in a liquid phase to be diesel oil and gasoline.

10. Separating: the diesel oil and gasoline are sent into the w/o separators (50), (51) to exclude water contained inside the diesel oil and gasoline, and then the purified diesel oil and gasoline are transported to a diesel oil tank (60) and gasoline tank (61) for storage. An air-extracting apparatus (54) is communicated with the diesel oil tank (60) and the gasoline tank (61) to decompress the pressure inside the tanks (60), (61).

According to the above description, the plastic waste is finally inverted to be multiple fuel materials such as gas, gasoline, and diesel oil, and other byproducts such as hydrochloride acid and active carbon. The fuel materials are selectively used in the heater (13) and heating furnace (41) to supply heat in self-sufficiency or used in other devices to supply energy. Therefore, the plastic waste is efficiently inverted in a beneficial way to achieve excellent exploitation.

Although the invention has been explained in relation to its preferred embodiment, it is to be understood that many other possible modifications and variations can be made without departing from the spirit and scope of the invention as hereinafter claimed.

What is claimed is:

1. A method for decomposing plastic waste to produce fuel materials, the method comprising following acts:
preparing a raw material, wherein the plastic waste is mixed with at least one catalyst to compose a raw material;
cracking, wherein the raw material is decomposed into cracked gas and cinder at high temperatures;
first fractionation, wherein the cracked gas gradually cooled to condense heavy gas oil with water, and then a residuum gas of the cracked gas after fractionating is separated from the heavy gas oil and water;
condensing, wherein the residuum gas is condensed to further separate surplus heavy gas oil from the gas;
removing hydrochloride acid, wherein the gas is drained to remove hydrochloride acid from the gas;
second fractionation, wherein the heavy gas oil is vaporized and extracted diesel gas and gasoline gas;
condensing, wherein the diesel gas and the gasoline gas both in a vapor phase are condensed to liquidize as a diesel oil and gasoline respectively in a liquid phase; and
separating, wherein the diesel oil and the gasoline in the liquid phase separated water from the diesel gas and the gasoline to be purified, and then the purified diesel oil and gasoline are respectively transported to a diesel oil tank (60) and a gasoline tank (61) for storage.

2. The method for decomposing plastic waste to produce fuel materials as claimed in claim 1, wherein the method further comprises the following act after the first fractionation:
separating oil and water, wherein the heavy gas oil and the water are separated.

3. The method for decomposing plastic waste to produce fuel materials as claimed in claim 1, wherein the method further comprises the following act after cracking:
filtering impurities, wherein the cracked gas is filtered to remove impurities in the cracked gas so as to avoid the impurities blocking pipes in the following acts.

4. The method for decomposing plastic waste to produce fuel materials as claimed in claim 1, wherein the cracking is controlled within a temperature range from 320° C.-360° C.

5. The method for decomposing plastic waste to produce fuel materials as claimed in claim 1, wherein first fractionation is controlled within a temperature range from 80° C.-360° C.

6. The method for decomposing plastic waste to produce fuel materials as claimed in claim 1, wherein the second fractionation is controlled within a temperature range from 100° C.-330° C.

7. Equipment for decomposing plastic waste to produce fuel materials, wherein the equipment comprises a cracking device (10) and the cracking device (10) comprises
an outer tank (11);
a heating tank (111) secured inside the outer tank (11);
a heater (13) secured at a bottom of the outer tank (11) to heat the cracking device (10); and
a cracking tank (12) mounted on the outer tank (11) for receiving and cracking the raw material.

8. Equipment for decomposing plastic waste to produce fuel materials, wherein the equipment comprises a water and oil separating tank (21) and the water and oil separating tank (21) comprises:
an outer tank (22);
an inner tank (23) secured inside the outer tank (22), wherein side walls of the inner tank (23) are lower than a height of the water and oil separating tower (21) so that the heavy gas oil and water overflow from the inner tank (23) over the side walls of the inner tank (23) to pour into the outer tank (22);
a recycling pipe (221) communicated with the outer tank (22) to re-fractionate heavy gas oil and water inside the outer tank (22);
an oil outlet (222) communicated with the outer tank (22) at a high-liquid mark to drain heavy gas oil to store; and
a water outlet (231) communicating the inner tank (23) to drain water out to store.

9. Equipment for decomposing plastic waste to produce fuel materials as claimed in claim 8, wherein the equipment comprises a pump (26) and the pump (26) is secured in the recycling pipe (211) of the water and oil separating tank (21) to push heavy gas, oil and water to a top of the first fractionating tower (20) and make the heavy gas oil with water fluently flow.
